(19) United States
(12) Patent Application Publication
(10) Pub. No.: US 2017/0360292 A1
(43) Pub. Date: Dec. 21, 2017

(54) DEVICE AND METHOD FOR SELF-RECOGNITION OF EYE OPACITIES

(71) Applicant: PORFIRIO VADILLO REIZABAL, CARACAS (VE)

(72) Inventor: PORFIRIO VADILLO REIZABAL, CARACAS (VE)

(21) Appl. No.: 15/186,967

(22) Filed: Jun. 20, 2016

(51) Int. Cl. A61B 3/02; A61B 3/00; A61B 90/00

(52) U.S. Cl. CPC A61B 3/02; A61B 3/0033; A61B 3/0008; A61B 2090/049; A61B 2560/0425

(57) ABSTRACT

A device and a method used for self-detection of eye opacities. The novel device utilizes a light source, which illuminates an orifice, to self-detect, measure, and locate an opacity's shadow projected on the retina using a grid sheet. This device monitors the early onset of eye disorders, such as cataracts, as well as post-operative detection of opacities after cataract surgery or capsulotomy. It is also portable, inexpensive, and does not require special training to utilize it.

### CROSS REFERENCE TO RELATED APPLICATIONS

Not Applicable

### FEDERALLY SPONSORED RESEARCH

Not Applicable

### SEQUENCE LISTING OR PROGRAM

Not Applicable

### REFERENCES CITED

ES 2 215 466 A1-Vadillo Reizabal, Porfirio
U.S. Pat. No. 3,967,885 A-Byler, William, H
U.S. Pat. No. 3,787,112 A-Lyons, J
U.S. Pat. No. 4,902,124-Roy Sr., Frederick H.
U.S. Pat. No. 3,903,870-Berndt Wolf-Dieter
U.S. Pat. No. 4,682,867-Gould, Herbert L.
U.S. Pat. No. 8,746,885-Ramesh Raskar

### BACKGROUND OF INVENTION

### Field of the Invention

The invention relates to a device that enables self-detection, measurement, and a method that aids in locating the shadows of opacities that are projected onto the retina, which may result in earlier recognition and later development, and permit the subsequent professional treatment of eye disorders, such as, but not limited to, eye cataracts. The inventive device may be used by the general public with no professional training in ophthalmology or related sciences. The invention more specifically relates to a hand held, portable, inexpensive device, that utilizes light diffraction travelling through a predetermined small orifice to the human eye and enables the viewer to contrast, compare, and measure eye opacities such that an observer having those opacities can visualize, measure, and graphically locate their position in the eye by using a grid sheet.

### Background

The behavior of light travelling through small orifices has been well studied and documented, and several applications resulted therefrom. These applications have helped develop photography, and have been used in ophthalmology to detect anomalies in eye vision; however, these techniques have not been directed towards the study of eye opacities. The invention is based on the unique behavior of the eye retina when it focuses on fixed eye opacities while operating in very low light conditions. Under such conditions, the retina is stretched to its maximum sensitivity capacity and it can distinguish eye opacities that under normal light conditions would remain unseen.

Searching the related prior art it could be found several patents related to the present invention. The most relevant prior art belongs to the present inventor (ES 2 215 466 A1-Vadillo Reizabal), hereinafter “ES Patent '466”, however while the objective of the inventions may be similar the current device is very different and not obvious, from the one disclosed before.

U.S. Pat. No. 3,967,885A-Byler, hereinafter “US Patent '885”, was alleged as prior art by the Spanish Patent Office as prior art in the prosecution of ES patent '466 but later were dismissed because the claims were unrelated and directed to a different device.

U.S. Pat. No. 3,787,112 A-Lyons, hereinafter “US Patent '112”, proposes a different device, more complex and voluminous. It requires additional optical devices, such as lenses, and does not utilize light diffraction since the apertures are much larger, in the order of 7 mm, approximately the aperture of the human pupil on low light conditions. With this aperture in the current inventive device, the eye opacities are undetectable. While US Patent '112 claims a reticular pattern screen for the projection of anomalies, it is not a condition of the present invention. While a claim is directed to the use of a screen, the lighting source of the screen is reflected light rather than light passing through a translucent screen. The difference in the results concerning the observation of eye opacities is remarkable and implies that the light source must be close to the eye and does not require amplification of the image.

U.S. Pat. No. 4,902,124-Roy, hereinafter “US Patent '124”, differs from the current invention insofar as the present invention proposes the possibility of multiple and selectable apertures, as each eye is different and at each stage of the development of opacities a different combination of parameters is needed. For example, and without limitation, at the earliest possible stage of a cataract the smallest orifice may be needed, since a larger diameter hole may result on the evanescence of the shadow.

Patents U.S. Pat. No. 3,903,870-Berndt, hereinafter “US Patent '870”, and U.S. Pat. No. 4,682,867-Gould, hereinafter “US Patent '867”, are related but claim a different device.

None of the aforementioned patents teaches how to use a lighted grid sheet to locate and position the observed eye opacities that frequently appear after cataract surgery; or the placement of the peripheral ring on an implanted lens; or the superposition of the eye lens and about other opacities that frequently appear over time in a progressive, but random location of the eye lens.

Even after surgery there is a possibility that some eye opacities were not removed. In some cases, it is necessary to dilate the pupil to observe the retina's peripheral area.

The possibility that a single apparatus will provide the ability to observe eye opacities of different dimensions and multiple orifices, at the earliest possible stage, by the person suffering from this malady and without professional help, has not yet been presented until the present invention. Further, it is possible that the various diffractions produced by opacities will enable the observer to determine the approximate distance from the opacity to the retina. Prior art also does not teach a method that differentiates between fixed and floating particles by “strong eye blinking.”

In summary, the following are the distinguishing features of the novel device that differentiate it from the cited prior art. The advantages of this novel device are that it is small, portable, inexpensive, and easy to build. It requires a small amount of energy to supply a light source. The device enables the observation of eye opacities after cataract surgery and capsulotomy: eye lens capsule, intraocular ring, and remaining opacities.

In contrast with U.S. Pat. No. 3,787,112, that presents a translucent reticulated foil, the present invention contains a reticulated observation area that is indirectly lighted through light reflection.

The present invention offers multiple apertures, which enables a more accurate selection according to the self-observer's sight capacity. The new invention allows for the observation of the pupil's reaction. This invention discloses a method to observe the fixed opacities by forceful eye blinking.

### SUMMARY OF THE INVENTION

The present invention discloses a novel device to self-detect eye opacities at the earliest possible stage, and allows for the measurement, and location of their shadows.

The device consists of a thin, opaque, film containing a plurality of selectable “very” small orifices. When high intensity light is screened through a select orifice, a luminous circle is observed, and within it, cataracts and other floating opacities are projected upon the eye retina.

### DESCRIPTION OF THE DRAWINGS

FIG. 1 is a view of one embodiment of invention.

FIG. 2 is a view of another embodiment of invention.

FIG. 3 is a view of another embodiment of invention.

### DETAILED DESCRIPTION OF THE INVENTION

The novel device permits the self-observation of opacities of the eye lens, such as cataracts, and in particular, post-cataract surgery eye opacities, laser capsulotomy, intraocular lens rings, and other eye opacities.

The principle behind the invention lies on observing fixed opacities in the retina of the eye, which involves viewing such opacities with minimal illumination through orifices at which instance the eye sensitivity is at its maximum. Notice: Fixed opacities cannot be observed under normal lighting (daylight) conditions.

In the early stages of opacity formation, the patient is unaware of the existence of such opacities. When the opacities mature and become larger, the disease becomes symptomatic where one may experience lack of brightness and definition in eyesight, for example in reading, whereupon at this stage the device serves minimal purpose. However, larger mature opacities can be viewed by replacing the smaller orifices with larger orifices.

An opaque thin sheet in which small orifices of varying diameters is central towards the functionality of this device. The lens and capsule of the eye are both observed through these small holes for the detection of opacities. These opacities are either fixed or movable, and differentiated by performing a simple test requiring the self-observer to blink repeatedly in order to determine which opacities are fixed in place.

This invention allows for the ability to detect the early onset of cataracts without having to wait until the disease becomes symptomatic. In addition, the simple utility of this device enables any self-observer to monitor the evolution of such orifices over time. Further, it is portable, inexpensive, and easy to build, with minimal light and energy requirements. Apart from detecting the early onset of cataracts, this device may serve to detect anomalies after cataract surgery and capsulotomy, such as the implanted intraocular lens ring and any remaining opacities.

In consideration of the three claims proposed, although varying modifications may be incorporated, its basic configurations will remain the same. In particular, the claims will maintain their control of light intensity, types of light frequency, size, and weight of the device(s), number and diameter of orifices, and light source. It is important to note that the diameter of the orifices are very critical because if they are too small, then the opacities will be difficult to differentiate. On the contrary, if they are too large (greater than one millimeter in diameter), the smaller opacities would be difficult to detect unless they have matured. Note: The eye light-guard used to limit the external light is not necessary for this determination.

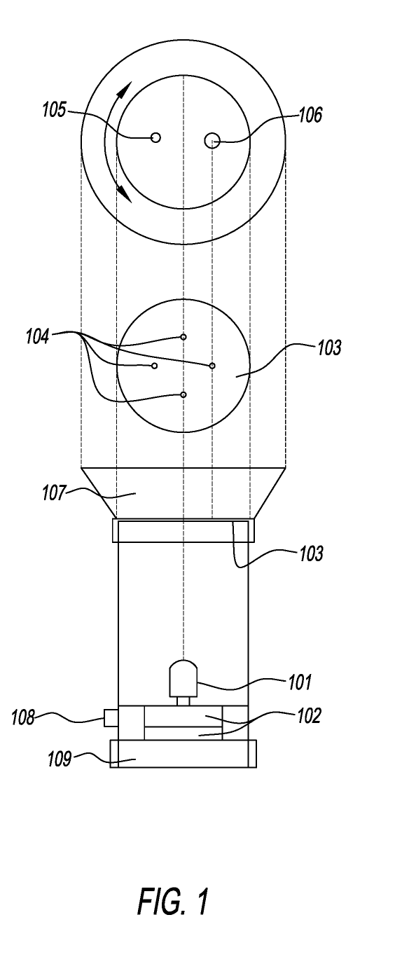

In addition to the proposed devices shown in FIGS. 1, 2, and 3, it will be possible to add electronic or mechanical control to vary the light source and/or aperture selection.

Among several light sources that may be used, a dimmable cell phone light provides a high intense light source that is portable and adjustable.

In addition, to observe the pupil reaction, the light intensity can be changed to view variations within the diameter of the luminous circle.

In FIG. 1 it is represented an embodiment of the present invention. It consists of a power source (102), such as for example and without limitation, a battery, a direct current source, photovoltaic generated energy, and the like, that powers light source (101), such as an LED or an incandescent lamp, that illuminates the display (103), which may be made of an opaque foil where there are two or more orifices (104) of different diameters of approximately one tenth of a millimeter. The orifices (104) are placed at the same radius from the physical center such that it will make it easy to select one by rotating a second foil. To diffuse the light, a translucent foil covers the orifices. You can add a rotating lamina (105) that is located on top of the display (103) coaxially containing a single hole (106) to rotate the screen hole (103), which would allow one to better observe the shadow of an opacity when looking through the orifices. A light-guard is also contained to limit external lighting (107). The device incorporates an external power switch (108) to connect the light source (101) to the power source (102), and a lid (109) to allow access to exchange batteries. The length of the device is not critical due to the large depth of field that have small holes to the passage of light; which is about 5 cm in length, with a diameter of approximately 1.5 centimeters.

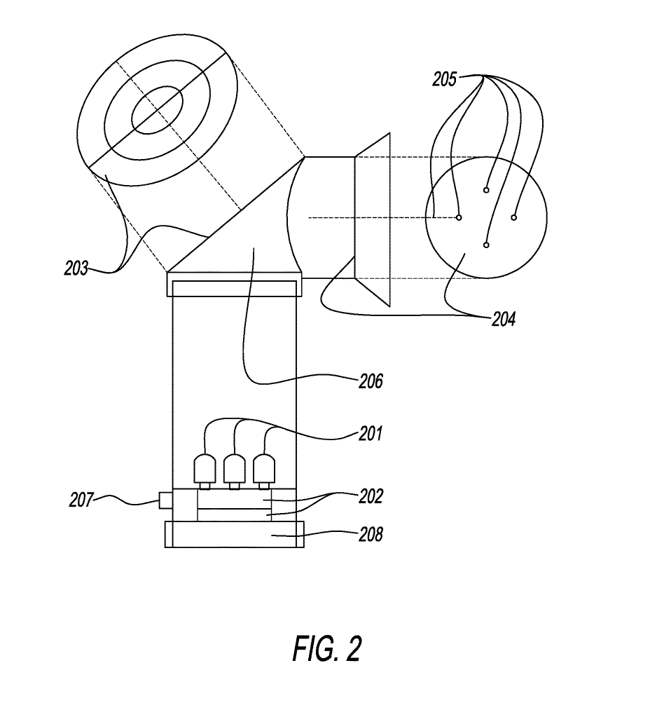

In FIG. 2 it is represented an alternative embodiment of the present invention. The most important difference with respect to the previous embodiment, represented in FIG. 1, is the incorporation of a reticular pattern to facilitate the positioning of the opacities.

The housing is similar to the previous FIG. 1 in relation to the light source (201) and the power source (202). The light source illuminates an elliptical cross-link (203), preferably inclined at 45 degrees in relation to the cross-section of the lantern; the reflected light incidents perpendicularly on the reticular pattern (204), where there are several orifices of varying diameters (205). By looking closely through the most suitable orifice an observer can see the opacities projected on the reticular pattern and thus facilitate their location on the grid and their drawing. While the reticular pattern may be elliptical, its projection on the eye is circular. The design corresponds to a simple calculation of projective geometry. The elliptical cross-linked (203) and the circular grid (204), as well as the eye light-guard, may be contained in an eye glasses frame (206) screwed into the cylinder on the top cover. It also contains an external power switch (207) to control the supply of energy and a lid cover (208) to facilitate access to the power supply.

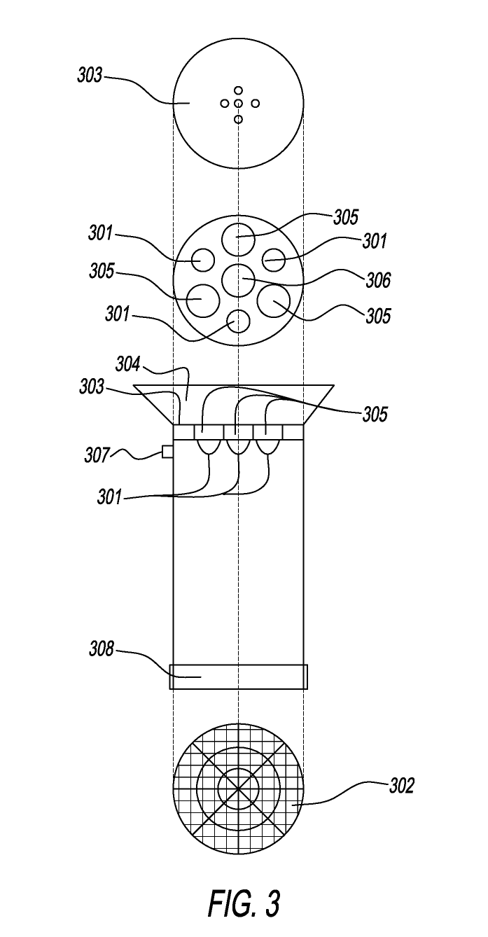

FIG. 3 represents yet another alternative embodiment of the present invention. The housing or casing is similar to the two previous figures but the reticular pattern is illuminated by reflection, and so it is advisable to place the light source and power source on top, near the holes.

The light source (301) illuminates the reticular pattern on the screen (302) positioned on the front cover; the light reflects and passes through the holes in the screen sets (303). It contains an eye light-guard to limit the external light (304). On the underside, there are several LEDs (301) arranged in a circular periphery and spaced evenly, powered with an appropriate amount of batteries (305). The central circular portion of the lamina (306) provides an area to observe orifices, which contains an external on/off switch (307). The front of the device contains a screw cap (308) that can be used to store several types of reticular patterns: rectangular, polar or hybrid. The light source (301), screen (303), and eye light-guard (304) jointly form a cap that can be removed for accessing the power supply and light sources. To obtain the best image of opacities, it is necessary that the self-observer stabilize the device by affixing it onto the face and using an eye light-guard.

What is claimed is:

1. A device to self-detect eye opacities, the device comprising:
a. a housing having a bottom end, a top end, and an enclosure connectively attached to, and respectively spacing, the bottom end from the top end;
b. an energy source disposed inside the housing and nearby the bottom end;
c. at least one light source, disposed inside the housing nearby bottom end;
d. an on-off power switch connectively connected and disposed in between the energy source and the at least one light source, capable of permitting and interrupting energy flow to the at least one light source;
e. a bottom screen disposed at the top end of the housing and having at least two orifices of varied diameters and disposed on a circular pattern;
f. a orifice selection screen disposed on top of the bottom screen connectively connected to the bottom screen through a center pin permitting the orifice selection screen to rotate around the center pin and having a small orifice that it is larger than the largest orifice of the at least two orifices of the bottom screen and it is disposed at the same radius as the bottom screen at least two orifices; and
g. an eye light-guard.

2. A device according to claim 1 whereby the energy source is one selected from a group consisting of at least one battery, an electrical current source, and photovoltaic cells.

3. A device according to claim 1 whereby the light source is one selected from a group consisting of incandescent lights, sunlight, electroluminescent lights, gas discharge light, and Light Emitting Diodes.

4. A device according to claim 1 whereby the housing has a cylindrical form.

5. A device to self-detect and determine the position of an opacity's shadow projected on the retina, the device comprising:
a. a housing having a bottom end, a top end, and an enclosure having a bottom segment contiguously connected to a elbow segment, disposed at an angle with respect to the bottom segment, contiguously connected to a top segment, disposed at an angle with respect to the elbow segment, the enclosure connectively attached to, and respectively spacing, the bottom end from the top end;
b. an energy source disposed inside the housing and nearby the bottom end;
c. at least one light source, disposed inside the housing nearby the bottom end;
d. an on-off power switch connectively connected and disposed in between the energy source and the at least one light source, capable of permitting and interrupting energy flow to the at least one light source;
e. a bottom screen disposed at the top end of the housing and having at least two orifices of varied diameters and disposed on a circular pattern;
f. a orifice selection screen disposed on top of the bottom screen connectively connected to the bottom screen through a center pin permitting the selection top screen to rotate around the center pin and having a small orifice that is larger than the largest orifice of the at least two orifices of the bottom screen and it is disposed at the same radius as the bottom screen at least two orifices;
g. a reflective inner wall engraved with a reticular pattern disposed on the elbow segment, whereby the reflective inner wall is positioned such that the light emanating from the at least one light source hits the reflective inner wall and deflects it towards the bottom screen; and
h. an eye light-guard.

6. A device according to claim 5 whereby the energy source is one selected from a group consisting of at least one battery, an electrical current source, and photovoltaic cells.

7. A device according to claim 5 whereby the light source is one selected from a group consisting of incandescent lights, sunlight, electroluminescent lights, gas discharge light, and Light Emitting Diodes.

8. A device according to claim 5 whereby the housing has a cylindrical form.

9. A device according to claim 5 whereby the reflective inner wall is angled, with respect to the bottom and top ends, at forty-five degrees.

10. A device to self-detect and determine the position of an opacity's shadow projected on the retina, the device comprising:
a. a housing having a bottom end, a top end, and an enclosure connectively attached to, and respectively spacing, the bottom end from the top end;
b. an energy source disposed inside the housing;
c. at least one light source, disposed inside the housing on the top end;
d. an on-off power switch connectively connected and disposed in between the energy source and the at least one light source, capable of permitting and interrupting energy flow to the at least one light source;
e. a bottom screen, forming the top end of the housing, having at least two orifices of varied diameters and disposed on a circular pattern;
f. a orifice selection screen disposed on top of the bottom screen connectively connected to the bottom screen through a center pin permitting the selection top screen to rotate around the center pin and having a small orifice that is larger than the largest orifice of the at least two orifices of the bottom screen and it is disposed at the same radius as the bottom screen at least two orifices;
g. a reflective inner wall engraved with a reticular pattern disposed on the bottom end, whereby the reflective inner wall is positioned such that the light emanating from the at least one light source hits the reflective inner wall and deflects it towards the bottom screen; and
h. an eye light-guard.

11. A device according to claim 10 whereby the energy source is one selected from a group consisting of at least one battery, an electrical current source, and photovoltaic cells.

12. A device according to claim 10 whereby the light source is one selected from a group consisting of incandescent lights, sunlight, electroluminescent lights, gas discharge light, and Light Emitting Diodes.

13. A device according to claim 10 whereby the housing has a cylindrical form.

14. A method to self-detect eye opacities, the method comprising:
a. utilizing a device, the device comprising:
i. a housing having a bottom end, a top end, and an enclosure connectively attached to, and respectively spacing, the bottom end from the top end;
ii. an energy source disposed inside the housing and nearby the bottom end;
iii. at least one light source, disposed inside the housing nearby bottom end;
iv. an on-off power switch connectively connected and disposed in between the energy source and the at least one light source, capable of permitting and interrupting energy flow to the at least one light source;
v. a bottom screen disposed at the top end of the housing and having at least two orifices of varied diameters and disposed on a circular pattern;
vi. a orifice selection screen disposed on top of the bottom screen connectively connected to the bottom screen through a center pin permitting the orifice selection screen to rotate around the center pin and having a small orifice that is larger than the largest orifice of the at least two orifices of the bottom screen and it is disposed at the same radius as the bottom screen at least two orifices; and
vii. an eye light-guard;
b. turning the on-off power switch such that energy flows to the at least one light source and the at least one light source is illuminated;
c. rotating the orifice selection screen such that its orifice aligns and corresponds with one of the at least two pinholes of the bottom screen;
d. placing an eye light-guard such that the eye is closely positioned and aligned with the center of the orifice selection screen's orifice;
e. observing a luminous circle corresponding to the aperture of the eye's pupil;
f. observing eye's fixed and moving particles and opacities;
g. rotating the orifice selection screen such that it corresponds and aligns with one of the at least two bottom screen's orifices that provides the most suitable observation of the eye opacities;
h. forcefully blinking the eye; and
i. observing the fixed opacities.

15. A method claim according to claim 14 whereby the at least one light source has a variable intensity luminosity

16. A method Claim according to claim 15 whereby the luminosity of the at least one light source is varied such that the observation of the eye opacities is sharpest.

17. A method to self-detect and determine the position of an opacity's shadow projected on the retina, the method comprising:
a. utilizing a device, the device comprising:
i. a housing having a bottom end, a top end, and an enclosure having a bottom segment contiguously connected to a elbow segment, disposed at an angle with respect to the bottom segment, contiguously connected to a top segment, disposed at an angle with respect to the elbow segment, the enclosure connectively attached to, and respectively spacing, the bottom end from the top end;
ii. an energy source disposed inside the housing and nearby the bottom end;
iii. at least one light source, disposed inside the housing nearby the bottom end;
iv. an on-off power switch connectively connected and disposed in between the energy source and the at least one light source, capable of permitting and interrupting energy flow to the at least one light source;
v. a bottom screen disposed at the top end of the housing and having at least two orifices of varied diameters and disposed on a circular pattern;
vi. a orifice selection screen disposed on top of the bottom screen connectively connected to the bottom screen through a center pin permitting the selection top screen to rotate around the center pin and having a small orifice that is larger than the largest orifice of the at least two orifices of the bottom screen and it is disposed at the same radius as the bottom screen at least two orifices;
vii. a reflective inner wall engraved with a reticular pattern disposed on the elbow segment, whereby the reflective inner wall is positioned such that the light emanating from the at least one light source hits the reflective inner wall and deflects it towards the bottom screen;
viii. an eye light-guard;
b. turning the on-off power switch such that energy flows to the at least one light source and the at least one light source is illuminated;
c. rotating the orifice selection screen such that its orifice aligns and corresponds with one of the at least two orifices of the bottom screen;
d. placing an eye on the eye light-guard such that the eye is closely positioned and aligned with the center of the orifice selection screen's orifice;
e. observing a luminous circle corresponding to the aperture of the eye's pupil;
f. observing eye's fixed and moving particles and opacities;
g. rotating the orifice selection screen such that it corresponds and aligns with one of the at least two bottom screen's orifices that provides the most suitable observation of the eye opacities;
h. observing the position of the eye's fixed and moving particles and opacities with respect to the reticular pattern;
i. noting the position of the eye's fixed and moving particles and opacities with respect to the reticular pattern;
j. forcefully blinking the eye;
k. observing the fixed opacities; and
l. noting the position of the eye's fixed opacities with respect to another reticula with the date of observation.

18. A method claim according to claim 17 whereby the at least one light source has a variable intensity luminosity.

19. A method Claim according to claim 18 whereby the luminosity of the at least one light source is varied such that the observation of the eye opacities is sharpest.

20. A method to self-detect and determine the position of an opacity's shadow projected on the retina, the method comprising:
a. utilizing a device, the device comprising:
i. a housing having a bottom end, a top end, and an enclosure connectively attached to, and respectively spacing, the bottom end from the top end;
ii. an energy source disposed inside the housing;
iii. at least one light source, disposed inside the housing on the top end;
iv. an on-off power switch connectively connected and disposed in between the energy source and the at least one light source, capable of permitting and interrupting energy flow to the at least one light source;
v. a bottom screen, forming the top end of the housing, having at least two orifices of varied diameters and disposed on a circular pattern;
vi. a orifice selection screen disposed on top of the bottom screen connectively connected to the bottom screen through a center pin permitting the selection top screen to rotate around the center pin and having a small orifice that is larger than the largest orifice of the at least two orifices of the bottom screen and it is disposed at the same radius as the bottom screen at least two orifices;
vii. a reflective inner wall engraved with a reticular pattern disposed on the bottom end, whereby the reflective inner wall is positioned such that the light emanating from the at least one light source hits the reflective inner wall and deflects it towards the bottom screen;
viii. an eye light-guard;
b. turning the on-off power switch such that energy flows to the at least one light source and the at least one light source is illuminated;
c. rotating the pinhole selection screen such that its orifice aligns and corresponds with one of the at least two orifices of the bottom screen;
d. placing an eye on the eye light-guard such that the eye is closely positioned and aligned with the center of the orifice selection screen's orifice;
e. observing a luminous circle corresponding to the aperture of the eye's pupil;
f. observing eye's fixed and moving particles and opacities;
g. rotating the orifice selection screen such that it corresponds and aligns with one of the at least two bottom screen's orifices that provides the most suitable observation of the eye opacities;
h. observing the position of the eye's fixed and moving particles and opacities with respect to the reticular pattern;
i. noting the position of the eye's fixed and moving particles and opacities with respect to the reticular pattern;
j. forcefully blinking the eye;
k. observing the fixed opacities; and
l. noting the position of the eye's fixed opacities with respect to another reticula with the date of observation.

21. A method claim according to claim 20 whereby the at least one light source has a variable intensity luminosity

22. A method Claim according to claim 21 whereby the luminosity of the at least one light source is varied such that the observation of the eye opacities is sharpest.
